(19) United States
(12) Patent Application Publication
(10) Pub. No.: US 2022/0071113 A1
(43) Pub. Date: Mar. 10, 2022

(54) Method for Establishing Submerged Vegetation System in Eutrophic Water Body

(71) Applicant: CHINESE RESEARCH ACADEMY OF ENVIRONMENTAL SCIENCES, Beijing (CN)

(72) Inventors: Caole LI, Beijing (CN); Jiaqian Li, Beijing (CN); Xiaoguang Li, Beijing (CN); Wei Li, Beijing (CN); Guowen Li, Beijing (CN); Lieyu Zhang, Beijing (CN); Chen Zhao, Beijing (CN)

(21) Appl. No.: 17/239,634

(22) Filed: Apr. 25, 2021

(30) Foreign Application Priority Data
Sep. 7, 2020 (CN) 2020109264814

(51) Int. Cl. A01G 33/00

(52) U.S. Cl. CPC A01G 33/00; C02F 3/327

(57) ABSTRACT

The present disclosure relates to a water environment governance technology, and particularly discloses a method for regulating primary productivity in a eutrophic water body. Emerging plants are cultivated in a water area to be governed to improve anti-wind wave and anti-water flow capabilities of the water area to be governed and establish a relatively stable microenvironment; and a modified clay molecular sieve, phosphorus-accumulating bacteria, grass seeds of submerged plants and silty clay are used to prepare a modified clay molecular sieve ecological base, and the modified clay molecular sieve ecological base is uniformly added to the water body of the area to be governed to inhibit the emission of phosphorus in the deposit and reduce disturbance, so that the cyanobacteria in the deposit is fixed, thereby improving the oxygen-deficient microenvironment of a sediment-water interface and achieving the objective of inhibiting growth of cyanobacteria.

### CROSS REFERENCE TO RELATED APPLICATION

This application claims the benefit of Chinese Patent Application No. 2020109264814, filed Sep. 7, 2020, which is hereby incorporated by reference herein in its entirety.

### TECHNICAL FIELD

The present disclosure relates to a water environment governance technology, in particular to a method for regulating primary productivity in a eutrophic water body.

### BACKGROUND

At present, cyanobacterial blooms and aquatic vegetation decline in eutrophic water bodies are water pollution problems that are faced by the world. Aquatic vegetation, especially submerged vegetation, as an important primary producer of lake ecosystems, can reduce the nutritive salt load of lake water bodies, control the growth of algae maintain clear water stable state and higher biodiversity of the water bodies, and its ecological reconstruction or restoration is considered to be one of the important measures for ecological restoration of lakes.

In the process of submerged vegetation restoration, restricted by environmental conditions such as poor underwater light conditions, high nitrogen and phosphorus nutritive salts, soft bottom sediment, insufficient supply of dissolved oxygen in the water bodies, and the like, it is difficult for the submerged vegetation to survive in these water bodies. In addition, dormant cyanobacteria in a water body deposit are the main source for the outbreak of the cyanobacteria in summer. Without intervention, with the reproduction of remaining submerged plants, the microenvironment of a sediment-water interface will be in a more oxygen-deficient state, which will create a favorable environment for the cyanobacteria in the deposit to float up into the water body. At the same time, the oxygen-deficient microenvironment will accelerate the emission of phosphorus in the deposit, which also provides favorable conditions for the outbreak of the cyanobacteria.

Therefore, there is an urgent need to provide a method for reestablishing a submerged vegetation system in a eutrophic water body. Improving the oxygen-deficient environment at the sediment-water interface and reducing the dormant cyanobacteria entering the water body are urgent problems to be solved.

### SUMMARY

In order to solve the problems in the prior art, in view of cyanobacterial blooms and aquatic vegetation decline caused by eutrophication of water bodies, the present disclosure provides a method for establishing a submerged vegetation system in a eutrophic water body to reestablish a healthy water body ecosystem.

In order to realize the objective of the present disclosure, the technical solution of the present disclosure is as follows:

A method for establishing a submerged vegetation system in a eutrophic water body includes:

(1) cultivating emerging plants in a water area to be governed to improve anti-wind wave and anti-water flow capabilities of the water area to be governed and establish a relatively stable microenvironment; and

(2) using a modified clay molecular sieve, phosphorus-accumulating bacteria, grass seeds and silty clay to prepare a modified clay molecular sieve ecological base, and uniformly adding the modified clay molecular sieve ecological base to the water body of the area to be governed.

Further, the grass seeds are grass seeds of submerged plants, preferably the grass seeds of Potamogeton crispus and Vallisneria spiralis. Considering the effects of light and water body turbidity, the grass seed mixing ratio is set to 2:1.

Further, a preparation method of the modified clay molecular sieve ecological base includes: weighing the modified clay molecular sieve and the silty clay in a ratio of 1:1, adding the grass seeds and the phosphorus-accumulating bacteria, and uniformly mixing the mixture.

The addition amount of the grass seeds is 40 to 60 seeds/m², that is, based on a unit area covered by the modified clay molecular sieve ecological base added to water, 40 to 60 grass seeds are added to the modified clay molecular sieve ecological base covering 1 square meter of the water area. The addition amount of the phosphorus-accumulating bacteria is 50% (v/v) relative to the total volume of the modified clay molecular sieve and the silty clay.

A preparation method of the modified clay molecular sieve includes: selecting clay including water body sediment and shore, and using the clay after drying, grinding and sieving, or purchasing a professional clay sewage treatment agent; and adding the treated clay to a chitosan solution to form a slurry, or spraying the chitosan solution on the constantly stirred clay (referring to CN 102502969A), where the amount of the chitosan is 1% to 1.5% (w/w) of the clay.

The phosphorus-accumulating bacteria are commercially available products, for example, anaerobic phosphorus-accumulating bacteria that can be purchased from Yangzhou Haicheng Biotechnology Co., Ltd. The product is in the form of bacterial powder, and the content of the phosphorus-accumulating bacteria is up to 95% or above.

Further, the first addition amount of the modified clay molecular sieve ecological base is not less than 500 g/m².

The modified clay molecular sieve ecological base is supplementally added every 5 to 7 days after the first addition, and the supplemental addition amount is 50% of the first addition amount.

Furthermore, the cultivation density of the emerging plants is 5 to 6 plants/m².

The emerging plants are respectively selected from one or more of Pontederia cordata, Typha orientalis and Canna indica and one or more of native organisms.

The cultivating time of the emerging plants needs to be before the recovery of the cyanobacteria.

The method of the present disclosure is applicable to the water area with a water depth of less than 3 meters and a wind speed of less than 8 m/s.

The phosphorus-accumulating bacteria of the present disclosure, also called phosphorus-absorbing bacteria and phosphorus-removing bacteria, are a special type of bacteria in a traditional activated sludge technique. The phosphorus-accumulating bacteria can inhale excessive amounts of phosphorus in sewage in an aerobic state, so that the phosphorus content in the phosphorus-accumulating bacteria exceeds the phosphorus content in ordinary bacteria by several times. This type of bacteria is widely used for biological phosphorus removal.

The raw materials or reagents involved in the present disclosure are all common commercial products, and the operations involved are conventional operations in the art unless otherwise specified.

On the basis of conforming to common knowledge in the art, the above preferred conditions can be combined with each other to obtain specific implementations.

The present disclosure has the following beneficial effects:

in the present disclosure, the modified clay molecular sieve ecological base is prepared to ensure a growth environment of the grass seeds of the submerged plants. The modified clay molecular sieve in the ecological base is a silicate compound, its porous structure is conducive to the growth of the phosphorus-accumulating bacteria, and at the same time, the modified clay molecular sieve improves the water-deposit microenvironment and increases the dissolved oxygen concentration in the microenvironment. Ferrous ions in the deposit are oxidized to ferric iron, thereby further inhibiting the emission of the phosphorus in the deposit. Meanwhile, the porous structure of the modified clay molecular sieve is combined with the growth of the submerged plants, thereby significantly reducing the disturbance at the water-deposit interface, and inhibiting the emission of the deposit cyanobacteria to the water body.

After the submerged vegetation system is established, the vigorous growth of the submerged plants can fix the sediment and decelerate a nutritive salt cycle at the sediment-water interface. At the same time, under the coordination of phenolic acid and fatty acid allelochemicals such as N-phenyl-2-naphthylamine, methyl dehydroabietate and ethyl dehydroabietate secreted by the submerged plants, cyanobacteria cells are oxidized in response to oxidative stress, thereby destroying lipid proteins and nucleic acids of the cyanobacteria cells and affecting the metabolism of the cyanobacteria. In combination with the modified clay molecular sieve ecological base improving the aerobic microenvironment of the water-deposit interface, the objective of inhibiting recovery, growth and reproduction of the cyanobacteria is achieved.

By using the method of the present disclosure, the germination rate of the grass seeds of the submerged plants can be increased to 80% or above, the field planting survival rate of aquatic plants is increased by 40% as compared with a traditional method, the emission of sediment pollution is effectively controlled, the submerged plant ecology improves and regulates the primary productivity of the water body, and the allelopathy of the plants further restricts the recovery, the growth and the reproduction of the cyanobacteria. In addition, the establishment of the submerged vegetation system provides a habitat for aquatic animals, forming a healthy water ecosystem.

### DETAILED DESCRIPTION

The preferred implementations of the present disclosure will be described in detail below in conjunction with embodiments. It should be understood that the following embodiments are given for illustrative purposes only, and are not intended to limit the scope of the present disclosure. Those skilled in the art can make various modifications and substitutions to the present disclosure without departing from the objective and spirit of the present disclosure.

Experimental methods used in the following embodiments are conventional methods unless otherwise specified.

Materials, reagents and the like used in the following embodiments are commercially available unless otherwise specified.

### Embodiment 1

An in-situ enclosure experiment was established in a laboratory. The simulated water depth was 1.0 to 2.0 m, and nutrients such as cod, N, P and the like were added such that a water body reached a eutrophic level. An experimental system was composed of 9 PVC permeable enclosures (5 m×2.5 m), and the bottom edges of enclosure cloths were buried with gabions to prevent the exchange of water bodies inside and outside the enclosures, and were fixed in the water body with steel pipes. Before the start of the experiment, all the enclosure cloths were submerged in water and allowed to stand for two weeks until the water bodies inside and outside the enclosures were fully balanced.

Three treatment measures were taken in the experiment, and three enclosures were adopted to repeat the experiment for each treatment.

Group A was a control group (not treated).

Group B was a modified clay molecular sieve ecological base: weighing a modified clay molecular sieve and silty clay in a ratio of 1:1, adding grass seeds and phosphorus-accumulating bacteria, and uniformly mixing a mixture. The addition amount of the grass seeds was 40 to 60 seeds/m², and the grass seeds were Potamogeton crispus seeds and Vallisneria spiralis seeds in a number ratio of 2:1. The addition amount of the phosphorus-accumulating bacteria was 50% (v/v) relative to the total volume of the modified clay molecular sieve and the silty clay.

A preparation method of the modified clay molecular sieve included: selecting clay including water body sediment and shore, and using the clay after drying, grinding and sieving, or purchasing a professional clay sewage treatment agent; and adding the treated clay to a chitosan solution to form a slurry, or spraying the chitosan solution on the constantly stirred clay (referring to CN 102502969A), where the amount of the chitosan was 1%-1.5% (w/w) of the clay.

The phosphorus-accumulating bacteria were commercially available products, for example, anaerobic phosphorus-accumulating bacteria that can be purchased from Yangzhou Haicheng Biotechnology Co., Ltd. The product was in the form of bacterial powder, and the content of the phosphorus-accumulating bacteria was up to 95% or above.

The first addition amount of the modified clay molecular sieve ecological base was not less than 500 g/m². The modified clay molecular sieve ecological base was supplementally added every 5 to 7 days after the first addition, and the supplemental addition amount was 50% of the first addition amount.

In Group C, only the grass seeds were added: the addition amount of the grass seeds was 40 to 60 seeds/m² (40 to 60 grass seeds were added per square meter of a water area), and the grass seeds were the Potamogeton crispus seeds and the Vallisneria spiralis seeds in a number ratio of 2:1.

In the experiment, a draught fan was configured to simulate wind waves, the wind speed was set to 7 m/s, and the water temperature was controlled at 25 degrees, which was suitable for recovery and reproduction of the cyanobacteria, and the seedling density of the Vallisneria spiralis was 600 plants/enclosure. After the system ran for two months, the density of the cyanobacteria and the germination rate of submerged plants in the water were tested.

A phytoplankton net was configured to take a phytoplankton sample, the density of the cyanobacteria was counted with a microscope, and at the same time, submerged plant seedlings were counted to calculate the germination rate of the seeds.

The results showed that compared with Group A, the number of the cyanobacteria in Group B was decreased by 80%, the germination rate of submerged plant seeds reached 82.5%, and the germination rate of the seeds was increased by 50%, so the effect was significant; and compared with group C, the number of the cyanobacteria in Group B was decreased by 70%, and the germination rate of the seeds was increased by 30%.

### Embodiment 2

An in-situ enclosure experiment was established in a laboratory. The simulated water depth was 1.0 to 2.0 m, and nutrients such as cod, N, P and the like were added such that a water body reached a eutrophic level. An experimental system was composed of 6 PVC permeable enclosures (5 m×2.5 m), and the bottom edges of enclosure cloths were buried with gabions to prevent the exchange of water bodies inside and outside the enclosures and were fixed in the water body with steel pipes. Before the start of the experiment, all the enclosure cloths were submerged in water and allowed to stand for two weeks until the water bodies inside and outside the enclosures were fully balanced.

Two treatment measures were taken in the experiment, and three enclosures were adopted to repeat the experiment for each treatment.

Group A was a modified clay molecular sieve ecological base: the treatment was the same as Group B in Embodiment 1.

In Group B, no phosphorus-accumulating bacteria were added: compared with the modified clay molecular sieve ecological base in Group A in this embodiment, no phosphorus-accumulating bacteria components were added, and the other treatments were the same.

In the experiment, a draught fan was configured to simulate wind waves, the wind speed was set to 7 m/s, and the water temperature was controlled at 25 degrees, which was suitable for recovery and reproduction of cyanobacteria, and the seedling density of Vallisneria spiralis was 600 plants/enclosure. After the system ran for 2 months, the density of the cyanobacteria and the germination rate of submerged plants in water were tested.

A phytoplankton net was configured to take a phytoplankton sample, the density of the cyanobacteria was counted with a microscope, and at the same time, submerged plant seedlings were counted to calculate the germination rate of seeds.

The results showed that compared with Group B, the number of the cyanobacteria in Group A was decreased by 80% relative to the control, the germination rate of submerged plant seeds reached 82.5%, and the germination rate of the seeds was increased by 27% relative to Group B, so the effect was significant.

### Embodiment 3

An in-situ enclosure experiment was established in a laboratory. The simulated water depth was 1.0 to 2.0 m, and nutrients such as cod, N, P and the like were added such that a water body reached a eutrophic level. An experimental system was composed of 6 PVC permeable enclosures (5 m×2.5 m), and the bottom edges of enclosure cloths were buried with gabions to prevent the exchange of water bodies inside and outside the enclosures and were fixed in the water body with steel pipes. Before the start of the experiment, all the enclosure cloths were submerged in water and allowed to stand for two weeks until the water bodies inside and outside the enclosures were fully balanced.

Two treatment measures were taken in the experiment, and three enclosures were adopted to repeat the experiment for each treatment.

Group A was a modified clay molecular sieve ecological base: the treatment was the same as Group B in Embodiment 1.

In Group B, a formula of the ecological base was changed (to an ordinary molecular sieve): compared with the modified clay molecular sieve ecological base of Group A in this embodiment, the modified clay molecular sieve was replaced with the ordinary molecular sieve, and the other treatments were the same.

In the experiment, a draught fan was configured to simulate wind waves, the wind speed was set to 7 m/s, and the water temperature was controlled at 25 degrees, which was suitable for recovery and reproduction of cyanobacteria, and the seedling density of Vallisneria spiralis was 600 plants/enclosure. After the system ran for 2 months, the density of the cyanobacteria and the germination rate of submerged plants in water were tested.

A phytoplankton net was configured to take a phytoplankton sample, the density of the cyanobacteria was counted with a microscope, and at the same time, submerged plant seedlings were counted to calculate the germination rate of seeds.

The results showed that compared with Group B, the number of the cyanobacteria in Group A was decreased by 80% relative to the control, the germination rate of submerged plant seeds reached 82.5%, and the germination rate of the seeds was increased by 30% relative to Group B, so the effect was significant.

Although the present disclosure has been described in detail above with general descriptions and specific implementations, some modifications or improvements can be made based on the present disclosure, which is obvious to those skilled in the art. Therefore, all these modifications or improvements made without departing from the spirit of the present disclosure shall belong to the protection scope of the present disclosure.

1. A method for establishing a submerged vegetation system in a eutrophic water body, comprising:
cultivating emerging plants in a water area to be governed to improve anti-wind wave and anti-water flow capabilities of the water area to be governed; and
using a modified clay molecular sieve, phosphorus-accumulating bacteria, grass seeds and silty clay to prepare a modified clay molecular sieve ecological base, and uniformly adding the modified clay molecular sieve ecological base to the water body of the area to be governed.

2. The method according to claim 1, wherein the grass seeds are grass seeds of submerged plants, and wherein the submerged plants comprise Potamogeton crispus and Vallisneria spiralis.

3. The method according to claim 2, further comprising preparing the modified clay molecular sieve ecological base, comprising: weighing the modified clay molecular sieve and the silty clay in a ratio of 1:1 and adding the grass seeds and the phosphorus-accumulating bacteria, and uniformly mixing a mixture, wherein an addition amount of the grass seeds is 40 to 60 seeds/m², and an addition amount of the phosphorus-accumulating bacteria is 50% (v/v) relative to a total volume of the modified clay molecular sieve and the silty clay.

4. The method according to claim 1, wherein a first addition amount of the modified clay molecular sieve ecological base is not less than 500 g/m².

5. The method according to claim 4, wherein the modified clay molecular sieve ecological base is supplementally added every 5 to 7 days after the first addition, and a supplemental addition amount is 50% of the first addition amount.

6. The method according to claim 4, wherein a cultivation density of the emerging plants is between 5 and 6 plants/m².

7. The method according to claim 6, wherein the emerging plants are respectively selected from one or more of Pontederia cordata, Typha orientalis and Canna indica and one or more of native organisms.

8. The method according to claim 6, wherein the emerging plants needs are cultivated before recovery of the cyanobacteria.

9. The method according to claim 1, wherein a water depth of the water area is less than 3 meters and a wind speed of the water area is less than 8 m/s.
